(19) United States
(12) Patent Application Publication
(10) Pub. No.: US 2016/0214910 A1
(43) Pub. Date: Jul. 28, 2016

(54) Natural Gas Reactors and Methods

(71) Applicant: Forrest A. King, Ooltewah, TN (US)

(72) Inventor: Forrest A. King, Ooltewah, TN (US)

(21) Appl. No.: 15/007,411

(22) Filed: Jan. 27, 2016

Related U.S. Application Data
(60) Provisional application No. 62/108,220, filed on Jan. 27, 2015.

(51) Int. Cl. C07C 1/12; C01B 3/36

(52) U.S. Cl. CPC C07C 1/12; C01B 3/36; C07C 2523/46; C07C 2523/755; C07C 2521/04; C01B 2203/025; C01B 2203/062; C01B 2203/1241; C01B 2203/84

(57) ABSTRACT

A method of producing heat for industrial purposes such as power generation can use at least one, if not two exothermic reactions. First, methane may be produced from carbon dioxide and hydrogen in a reactor. This reaction produces heat. The methane may be burned, or oxidized (which is also an exothermic reaction) to produce carbon dioxide and hydrogen. Oxygen and/or hydrogen may supplement the process as could be produced from the electrolysis of water. Carbon dioxide may be obtained from a variety of sources.

### CLAIM OF PRIORITY

This application claims the benefit of U.S. Provisional Application No. 62/108,220 filed Jan. 27, 2015, which is included herein by reference, in its entirety.

### FIELD OF THE INVENTION

The present invention relates to a natural gas reactor and method of use whereby at least one, if not two reactors are utilized to generate heat and/or possibly produce component gasses for various uses.

### BACKGROUND OF THE INVENTION

Until recently, natural gas used to be relatively expensive. Accordingly, a need exists to produce relatively inexpensive natural gas under at least some conditions.

Furthermore, many industrial requirements require heat for various processes. Various food companies and other industries consume enormous amounts of natural gas which is used as a source of heat to manufacture potato chips and other snacks. What if this heat could be produced in a much more cost effective manner? The present method employed by some snack manufacturing plants is to burn natural gas. Accordingly, a need exists to produce heat for use in industrial or other environments while also possibly producing at least one of methane (CH₄), carbon dioxide and/or carbon monoxide and/or hydrogen.

### SUMMARY OF THE INVENTION

It is an object of many embodiments of the present invention to provide an improved heat producing and heating systems for industrial environments.

It is another object of many embodiments of the present invention to provide an improved method of producing methane from hydrogen and carbon dioxide.

It is another object of the present invention to provide an improved method of cyclically performing exothermic reactions in an effort to greatly enhance the heating capability from methane in environments, particularly when oxygen and/or hydrogen can be relatively inexpensively obtained.

Accordingly, the present invention relates to a first reactor which receives the inputs of carbon dioxide and hydrogen and then through a fusion reaction at temperatures such as about 500° Celsius in the presence of a catalyst, an exothermic reaction is conducted to produce methane gas CH₄ (i.e., natural gas) and water. In the testing conducted by the applicant, roughly 98% conversion was achieved.

Although a heater is required initially to heat the reactor to 500° Celsius, the reaction of the fusion of carbon dioxide with hydrogen is a self-sustaining exothermic reaction which was found to produce more than enough heat to maintain the temperature as well as provide an additional heat source that could be utilized for industrial environments. For instance, a food industry would require a temperature of roughly 350° Fahrenheit to fry potato chips. Accordingly, using the applicant's process there is a sufficient heat from this first process that could be utilized to provide at least some of the heat.

The carbon dioxide and hydrogen may come from various sources and they may either be waste products themselves or be provided relatively cost efficiently. For instance, the hydrogen may be produced through other technology of the applicant such as from electrolysis of water utilizing solar energy or other energy sources that could be extremely cost effective. One may also be able to find an inexpensive source of carbon dioxide such as from waste products of fermentation, oxidation of methane, products of combustion, or even being emitted from volcanos, hot springs or geysers or from the dissolution of water and various acids.

In fact, the applicant is working on methods to separate carbon dioxide from air which would provide an extremely inexpensive source of carbon dioxide.

Once the first process of producing methane gas and water is performed to produce methane, the methane could be burned for additional heat and/or alternatively, utilizing a partial oxidation reaction, effectively the process could be reversed so that the CH₄ and possibly steam and/or oxygen could be directed into the same or another reactor with a catalyst to provide an exothermic catalytic partial oxidation reaction and a water gas shift reaction so that the methane together with water and/or oxygen can shift to at least one portion of carbon monoxide and water and/or carbon dioxide and hydrogen and more heat. If the hydrogen and the carbon dioxide are not necessary for further industrial processes, the hydrogen and carbon dioxide can be run back through the first reactor to produce methane and water again thereby producing the first process i.e. the first exothermic reaction and more heat.

Assuming another 98% efficient conversion, the consumption of methane is roughly about 4%, through the cycle. In industrial or other environments, this could reduce the current usage of methane to produce heat by a factor of about 25 which could significantly lower the heating bills for a company to roughly 4% of their current expenses. After the capital costs of the reactor systems and heat exchangers are in place, roughly 4% of the current costs are expected for the same amount of heat. This would appear to be particularly attractive for many applications. Furthermore, in countries such as Japan, particularly if there is an excess supply of hydrogen and carbon dioxide in the marketplace, clean natural gas (i.e. methane) can be produced for power generation, heating and/or other purposes. Furthermore, the two cyclical reaction processes can be employed for various heating sources such as to generate power or other purposes.

### BRIEF DESCRIPTION OF THE DRAWINGS

The particular features and advantages of the invention as well as other objects will become apparent from the following description taken in connection with the accompanying drawings in which:

FIG. 1 is a schematic representation of the presently preferred embodiment of the present invention of a first portion of the invention, namely, the conversion of carbon dioxide and hydrogen to methane and water;

FIG. 2 is a schematic representation of the conversion of methane and oxygen and/or water to at least one of CO₂ and hydrogen if not carbon monoxide and/or water through the catalytic partial oxidation process;

FIG. 3 is a chemical representation of oxidation path of methane CH₄ to CO₂ and hydrogen;

FIG. 4 is a chemical representation of the fusion of carbon dioxide and hydrogen into methane and water; and

FIG. 5 is a schematic representation of an industrial system using the reactors for heat.

### DETAILED DESCRIPTION OF THE DRAWINGS

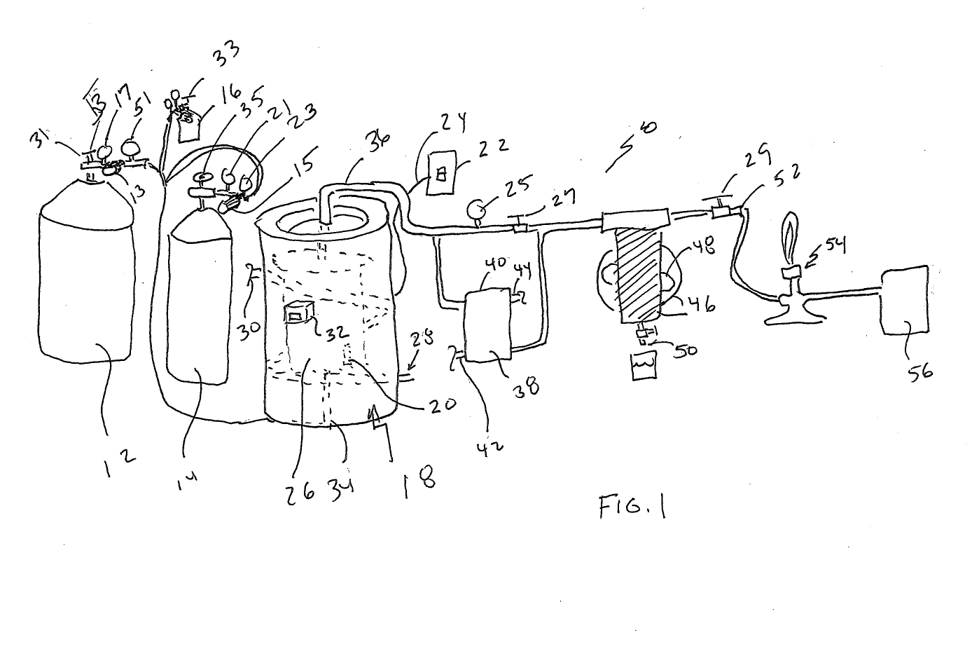

FIG. 1 shows a presently preferred embodiment of the present invention addressing in a first direction where carbon dioxide and hydrogen represented by CO₂₊H₂ as shown in first and second canisters 12,14 are combined to form methane CH₄ and water and energy (as an exothermic reaction). The carbon dioxide and hydrogen may come from different sources or be premixed such as provided in first canister 12 and/or second canister 14. For ease of use the applicant has a premixed carbon dioxide and hydrogen in the first canister 12 and provides nitrogen purge with second canister 14. Other embodiments may have yet another canister, separate such as canister 16 which could be similar or dissimilar to first canister 12 to provide a supply of hydrogen and/or carbon dioxide (opposite of first canister 12) to reactor 18 for the fusion reaction to occur.

The process of forming methane from CO₂ and hydrogen is often referred to as the Sabatier reaction or Sabatier process. When combined with the catalytic partial oxidation reaction shown in FIG. 4, two exothermic reactions can cyclically be provided.

The initial heat for the first process is optimally in a range of at least about 300°-400° Celsius and preferably occurs in the presence of a nickel catalyst 20. Once the desired starting temperature is achieved in the reactor 18 such as with a power supply illustrated as being provided switch 22 (such as providing electricity through power line 24 to heater 26), the reaction can begin. Once the reaction starts, the reactor 18 may continue to be brought up to temperature or temperature maintained, with the power secured from switch 22. The temperature of the reactor 18 can be maintained, and in fact give off extra heat such through heat exchanger represented by inlet 28 and outlet 30 which could direct the fluid through reactor 18 and take off extra heat to maintain the optimal temperature. The optimal pressure of the reactor 18 may also be maintained during this process.

While a nickel catalyst can be used, ruthenium on alumina may also be utilized as well as other catalysts which would be known to those of ordinary skill in the art. Hydrogen can be readily obtained from the electrolysis of water as one of ordinary skill in the art would understand. Carbon dioxide might be obtained from combustion processes, oxidation of methane from naturally occurring sources such as volcanic eruptions, geysers, etc., or it may also be extracted from air or fossil fuel waste such as by the amine process which is a scrubbing process normally used to remove hydrogen sulfide H₂S and carbon dioxide from gasses. CO₂ scrubbers are utilized in various applications.

Meter 32 can report the temperature inside the reactor 18 so that the operator will know when to begin the reaction once the desired temperature has been achieved and the flow of gasses can commence. The exothermic reaction in the reactor 18 can then begin to generate heat. The carbon dioxide and hydrogen are preferably directed through at least one inlet 34 or possibly through separate inlets into the reactor 18 to where the exothermic reaction in the present catalyst 20 occurs. Hot gasses are directed out outlet 36 where they can then proceed through a heat exchanger 38 which may have a meter 40 to advise of the temperature. Heat exchanger 38 may have inlet 42. Outlet 44 may provide a source of heat which may also be utilized for various purposes such as for power and/or heat purposes. Fluid may pass through a heat exchanger in the reactor 28 as represented by inlet 28 and outlet 30 as would be understood by those of ordinary skill in the art. Water can be turned into steam in one or both of these heat exchangers for use with other heating operations. Other fluids could be utilized with other embodiments. The exhaust gas from the reactor 18 can then proceed to a knock-out drum 46 or other heat exchanger so that water can be separated from methane. Cooling can be provided such as by fan 48 and/or a similar structure like a heat exchanger 38 or otherwise so that water for the reactor 18 can then be ejected such as from outlet 50 which has been found to be a particularly purified form of water. Methane can exit from outlet 52 and can either be burned such as with one or more burners 54 and/or stored in storage 56 as would be understood by those of ordinary skill in the art. This is reaction is exothermic in nature.

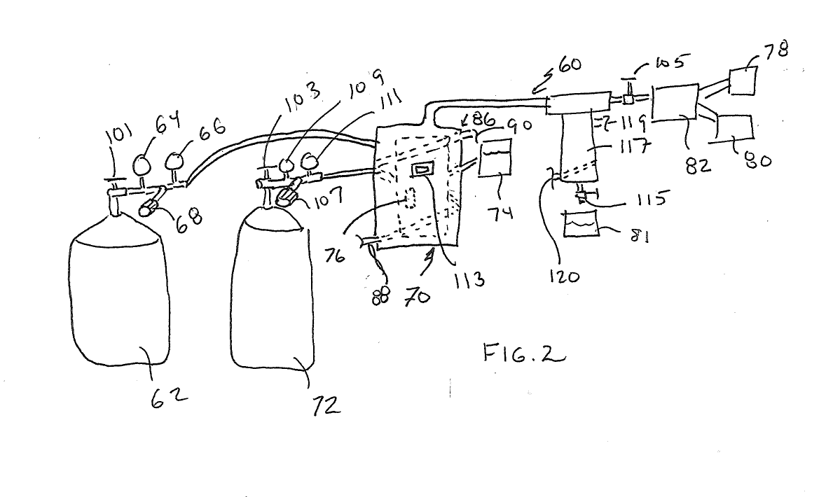

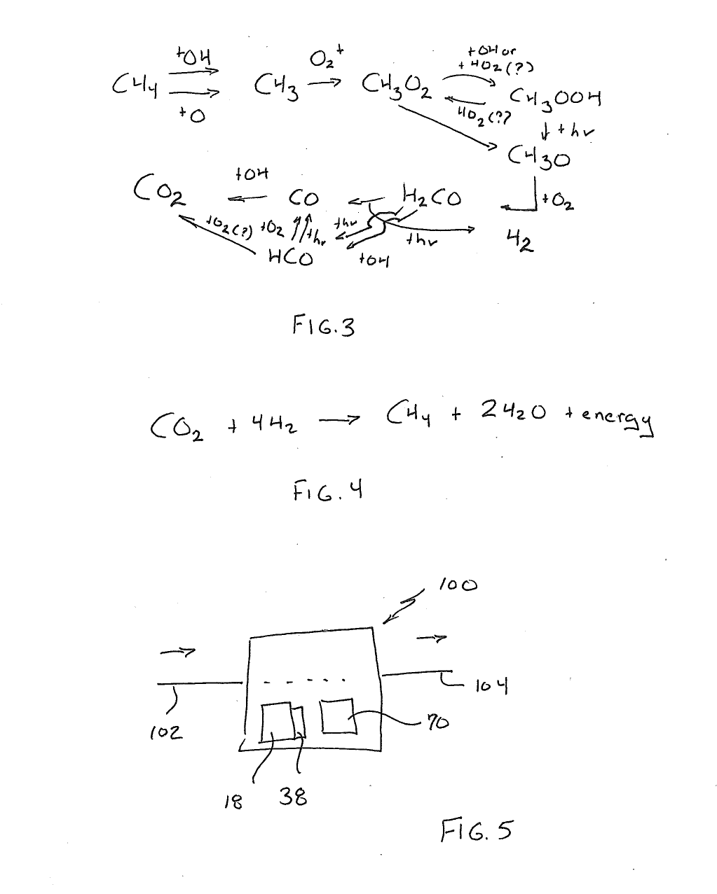

With the methane being stored, it can then be utilized in a separate process in a cyclical manner as shown by FIG. 2 to continue to generate even more heat. The second reaction can be provided by system 60 showing a methane supply 62 with pressure gauges 64,66 and regulator 68 and can direct a flow of methane into reactor 70 possibly along with a supply of oxygen 72 and/or steam or water 74 which may be obtained from the system 10 or otherwise. Oxygen may be obtained from a possible electrolysis reaction which the hydrogen was obtained for the first process or system 10 shown in FIG. 7. The oxygen 72 and/or hydrogen and/or methane can react with the catalyst 76 in the reactor 70 to ultimately form carbon dioxide 78 and hydrogen 80 is shown in FIG. 2 and/or alternatively form carbon monoxide 82 and/or water 81 and/or hydrogen 89.

It is preferred to continue the process to form the catalytic partial methane oxidation process from methane supply 52 all the way to carbon dioxide and hydrogen which is an exothermic reaction giving off heat such as to heat exchanger 86 represented by inlet 88 and outlet 90 for which such heat can be utilized to produce steam and/or heat for use in turbines for power generation and/or for heat in other heat exchangers. For instance, FIG. 5 shows a heater 100 comprised of reactors 18 and 70 as well as heat exchanger 38 and/or possibly others in an industrial setting such as a heater 100 receiving product or supply being provided on belt 102 as input and then being removed from belt 104 as output such as could occur in the cooking of potato chips and/or other processes. Of course, other industrial processes may use any one of the various heat exchangers 18 and/or reactors 18,38,70 and/or others as would be understood by those of ordinary skill in the art.

Referring back to FIG. 1, in the process the system 10 may employ a way to regulate pressure of the various gasses such as through the use of one or more regulators 13,15 and use of pressure gauges such as 17,51,21,23,25 and others. Various cutoff valves 27,29,53,33,35 and others may be useful for various embodiments for safety or other purposes.

Similarly, in FIG. 2 cutoff valves 101,103,105 and/or others may be utilized Regulator 68 and 107 may be utilized as well as pressure gauges 64,66,109,111 and/or others. Various temperatures can be monitored such as with meter 113. Water may be extracted in valve 115 or otherwise if at all, and of course, the knock-out drum 117 can have a heat exchanger with inlet 120 and outlet 119 as would be understood by those of ordinary skill in the art or could take other forms of heat exchangers.

By running the two exothermic reactions simultaneously within a heater 100, a large amount of heat can be produced. This heat can be provided for various processes. If the heat were utilized to heat in place of only the burning of natural gas, the applicant believes that with the efficiencies of being roughly 98% conversion in both directions, the total loss in completing the cycle would be roughly 4%. The amount of heat generated could be roughly about 25 times that of the amount if methane alone were simply burned. With the amount of methane roughly consumed by the process due to inefficiencies, this is 25 times less for the same amount of heat and is believed to be a huge improvement and cost savings over prior art. Other embodiments may not be this efficient but the applicant believes that the generation of at least about ten times as much heat as a traditional natural gas burner in terms of efficiency is relatively easily achieved. Some embodiments of this technology may achieve efficiencies closer to 25 times.

Certainly either of the two processes shown in FIGS. 1 and/or 2 can be run in a single direction for various embodiments, for instance, if there is an abundance of carbon dioxide and hydrogen on location, then the heat reaction could possibly be utilized for various purposes while the methane could be used for traditional natural gas applications. Similarly, if there is an abundance of methane, an ability to manufacture CO₂ and hydrogen gas for various purposes could be used, possibly while enjoying the heat for certain applications. At least one or both of these processes may be employed in a cyclical manner for use in generating steam or other heating applications such as heating buildings, heating water such as to steam for power generators, and/or providing other mechanical and/or chemical processes including the generation of various gasses.

Numerous alterations of the structure herein disclosed will suggest themselves to those skilled in the art. However, it is to be understood that the present disclosure relates to the preferred embodiment of the invention which is for purposes of illustration only and not to be construed as a limitation of the invention. All such modifications which do not depart from the spirit of the invention are intended to be included within the scope of the appended claims.

Having thus set forth the nature of the invention, what is claimed herein is:

1. A method of heat generation and producing methane comprising the steps of:
(a) providing hydrogen and carbon dioxide to a reactor;
(b) exothermically reacting the hydrogen and carbon dioxide in the reactor to form methane, water and heat;
(c) separating the methane from the water; and
(d) at least one of the following steps:
(i) using the heat from the reactor for an industrial process selected from the group of generating power in a turbine and heating;
(ii) burning the methane for an industrial process selected from the group of generating power in a turbine and heating; and
(iii) oxidizing the methane of step (b) in an exothermic reaction to produce at least carbon dioxide and hydrogen, at least one of which is used to repeat step (a) above, and heat, said heat used for an industrial process selected from the group of generating power in a turbine and heating.

2. The method of claim 1 wherein step (d) (iii) is performed and the oxidation step further produces carbon monoxide and water, with at least one of the carbon dioxide and hydrogen separated from the carbon monoxide.

3. The method of claim 2 wherein step (d) (iii) is performed and both the carbon dioxide and hydrogen are used to repeat step (a).

4. The method of claim 1 wherein step (d) (iii) is performed and both the carbon dioxide and hydrogen are used to repeat step (a).

5. The method of claim 1 further comprising a heat exchanger receiving output of the reactor, said heat exchanger used for an industrial process selected from the group of generating electricity and heating.

6. The method of claim 1 wherein the carbon dioxide provided for step (a) is:
(a) a waste product from one of
(i) combustion, and
(ii) fermentation;
(b) generated from dissolution of water and an acid;
(c) generated form an amine process from fossil fuels; and
(d) obtained from a natural emission from one of:
(i) geysers,
(ii) hot springs; and
(iii) volcanoes.

7. The method of claim 1 wherein the hydrogen provided for step (a) is generated from the step of electrolysis of water.

8. The method of claim 7 wherein the step of electrolysis performed generates oxygen, and the oxygen is used in step (d) (iii).

9. The method of claim 1 wherein the reactor has a heat exchanger for use with step (d) (i).

10. The method of claim 1 further comprising the step of providing a heater, said heater initially heating the reactor to at least 300 C to begin the exothermic reaction step, and then securing the heater while continuing the exothermic reaction step.

11. The method of claim 1 wherein the reactor has a catalyst selected from the group of nickel, ruthenium and alumina, and the exothermic reaction step utilizes the catalyst to assist in performing the reaction.

12. A method of heat generation and producing methane comprising the steps of:
(a) oxidizing methane in an exothermic reaction to produce heat and at least carbon dioxide and hydrogen, said heat used for an industrial process selected from the group of generating power in a turbine and heating;
(b) providing hydrogen and carbon dioxide (having the at least one from step (a)) to a reactor;
(c) exothermically reacting the hydrogen and carbon dioxide in the reactor to form methane, water and heat;
(d) separating the methane from the water; and
(e) at least one of the following steps:
(i) using the heat from the reactor for an industrial process selected from the group of generating electricity and heating; and
(ii) burning the methane for an industrial process selected from the group of generating electricity and heating.

13. The method of claim 12 wherein both the hydrogen and the carbon dioxide are provided to the reactor from the oxidizing step.

14. The method of claim 12 wherein when step (a) is performed and the oxidation step further produces carbon monoxide and water, with at least one of the carbon dioxide and hydrogen separated from the carbon monoxide.

15. The method of claim 12 wherein steps (a)-(e) are performed repeatedly in a cycle.

16. The method of claim 12 further comprising a heat exchanger receiving output of the reactor, said heat exchanger used for an industrial process selected from the group of generating electricity and heating.

17. The method of claim 12 wherein the carbon dioxide provided for step (a) is:
(a) a waste product from one of
(i) combustion, and
(ii) fermentation;
(b) generated from dissolution of water and an acid;
(c) generated form an amine process from fossil fuels; and
(d) obtained from a natural emission from one of:
(i) geysers,
(ii) hot springs; and
(iii) volcanoes.

18. The method of claim 12 wherein the reactor has a heat exchanger for use with step (d) (i).

19. The method of claim 12 further comprising the step of providing a heater, said heater initially heating the reactor to at least 300 C to begin the exothermic reaction step, and then securing the heater while continuing the exothermic reaction step.

20. The method of claim 12 wherein the reactor has a catalyst selected from the group of nickel, ruthenium and alumina, and the exothermic reaction step utilizes the catalyst to assist in performing the reaction.
